(19) United States
(12) Patent Application Publication
(10) Pub. No.: US 2024/0252794 A1
(43) Pub. Date: Aug. 1, 2024

(54) ROTATIONAL LOCK FOR DILATOR

(71) Applicant: Boston Scientific Scimed, Inc., Maple Grove, MN (US)

(72) Inventor: Jeffery D. Arnett, Louisa, VA (US)

(21) Appl. No.: 18/430,240

(22) Filed: Feb. 1, 2024

Related U.S. Application Data
(60) Provisional application No. 63/482,759, filed on Feb. 1, 2023.

(51) Int. Cl. A61M 29/00; A61M 25/00

(52) U.S. Cl. CPC A61M 29/00; A61M 25/0097; A61M 2210/125

(57) ABSTRACT

A dilator includes a dilator shaft defining a lumen for receiving a functional device, such as for example a puncture device, therethrough. The dilator shaft includes a proximal portion for manipulation by a user and a distal portion to placement in or near the heart. A dilator hub is coupled to the proximal portion of the dilator shaft and includes a rotational coupling structure for coupling to a corresponding hub of a sheath.

### CROSS REFERENCE TO RELATED APPLICATIONS

This application claims the benefit of U.S. Provisional Patent Application No. 63/482,759, filed Feb. 1, 2023, which is hereby incorporated in its entirety.

### TECHNICAL FIELD

The present invention relates generally to methods and devices usable to deliver a therapy to a patient. More specifically, the present invention is concerned with a system and method for delivering a therapy device to a heart.

### BACKGROUND

Devices currently exist for creating a puncture, channel, or perforation within a tissue located in a body of a patient. One such device is the Brockenbrough™ Needle, which is commonly used to puncture the atrial septum of the heart. This device is a stiff elongated needle, which is structured such that it may be introduced into a body of the patient via the femoral vein, and directed towards the heart. This device relies on the use of mechanical force to drive the sharp tip through the septum. Alternatively, radiofrequency perforation apparatuses have been developed, whereby the septal perforation is accomplished by the application of focused radiofrequency energy to the septal tissue via an electrode at the distal end of a relatively thin conductive probe.

Such perforation devices are often used in conjunction with a dilator to help support and guide the perforation device. Such dilators are often used in conjunction with a therapy sheath adapted to deliver a therapy to the patient.

### SUMMARY

An enhanced dilator includes a dilator shaft defining a lumen for receiving a functional device (e.g., a puncturing device) therethrough. The dilator shaft includes a proximal portion for manipulation by a user and a distal portion to placement in or near the heart. A dilator hub is coupled to the proximal portion of the dilator shaft and includes a rotational coupling structure for coupling to a corresponding hub of a therapy sheath.

Example 1 is a dilator for facilitating access to a patient's heart and for coupling with a sheath including a sheath hub. The dilator includes a dilator shaft defining a lumen adapted to receive and support a puncturing device. The dilator shaft includes a proximal portion for manipulation by a user and a distal portion for placement in or near the heart. A dilator hub is coupled to the proximal portion of the dilator shaft. The dilator hub includes a rotational coupling structure for coupling to the sheath hub so as to inhibit relative rotation between the dilator and the sheath. The dilator hub is configured to accommodate an insertion angle.

Example 2 is the dilator of Example 1, wherein the dilator hub is directly coupled to the dilator shaft.

Example 3 is the dilator of Examples 1 or 2, wherein the sheath is a therapy sheath and the puncturing device is an RF puncturing device.

Example 4 is the dilator of any of Examples 1-3, further comprising an axial lock feature configured to create a resistance to an axial disengagement force, such that the dilator hub is secured axially within the sheath hub.

Example 5 is the dilator of any of Examples 1-4, wherein the axial lock feature includes a protrusion adapted to mate with a shoulder.

Example 6 is the dilator of Example 5, wherein the protrusion is annular.

Example 7 is the dilator of any of Examples 1-6, wherein the dilator hub includes an angled disengagement surface adapted to contact a mating surface on the sheath hub.

Example 8 is the dilator of Example 7, wherein upon rotation of the dilator hub, the mating surface generates an axial disengagement force upon the disengagement surface.

Example 9 is the dilator of Example 8, wherein when the disengagement force becomes high enough to overcome an axial lock, relative motion in both axial and rotational directions occurs.

Example 10 is the dilator of Example 7, wherein the angled disengagement surface and the mating surface have a different angle.

Example 11 is the dilator of Example 7, wherein the angled disengagement and the mating surface have a same angle.

Example 12 is the dilator of any of Examples 1-11, wherein the rotational coupling structure includes a plurality of tapered surfaces.

Example 13 is the dilator of Example 12, wherein the sheath hub includes surfaces corresponding to the plurality of tapered surfaces.

Example 14 is the dilator of any of Examples 1-13, wherein the dilator hub is configured to accommodate an insertion angle.

Example 15 is the dilator of any of Examples 1-14, wherein the sheath hub has a tapered or funnel shape opening.

Example 16 is a dilator for facilitating access to a patient's heart and for coupling with a sheath including a sheath hub. The dilator includes a dilator shaft defining a lumen adapted to receive and support a puncturing device. The dilator shaft includes a proximal portion for manipulation by a user and a tapered distal portion for placement in or near the heart. A dilator hub is coupled to the proximal portion of the dilator shaft. The dilator hub includes a rotational coupling structure for coupling to the sheath hub so as to inhibit relative rotation between the dilator and the sheath. The dilator hub is configured to self-align into proper engagement with the sheath hub.

Example 17 is the dilator of Example 16, wherein the dilator hub is directly coupled to the dilator shaft.

Example 18 is the dilator of Example 16, wherein the sheath is a therapy sheath and the puncturing device is an RF puncturing device.

Example 19 is the dilator of Example 16, further comprising an axial lock feature configured to create a resistance to an axial disengagement force, such that the dilator hub is secured axially within the sheath hub.

Example 20 is the dilator of Example 16, wherein the axial lock feature includes a protrusion adapted to mate with a shoulder.

Example 21 is the dilator of Example 20, wherein the protrusion is annular.

Example 22 is the dilator of Example 16, wherein the dilator hub includes an angled disengagement surface adapted to contact a mating surface on the sheath hub.

Example 23 is the dilator of Example 22, wherein upon rotation of the dilator hub, the mating surface generates an axial disengagement force upon the disengagement surface.

Example 24 is the dilator of Example 23, wherein when the disengagement force becomes high enough to overcome an axial lock, relative motion in both axial and rotational directions occurs.

Example 25 is the dilator of Example 22, wherein the angled disengagement surface and the mating surface have a different angle.

Example 26 is the dilator of Example 22, wherein the angled disengagement and the mating surface have a same angle.

Example 27 is the dilator of Example 16, wherein the rotational coupling structure includes a plurality of tapered surfaces.

Example 28 is the dilator of Example 27, wherein the sheath hub includes surfaces corresponding to the plurality of tapered surfaces.

Example 29 is the dilator of Example 16, wherein the sheath hub has a tapered or funnel shape opening.

Example 30 is a system for facilitating access to a patient's heart. The system includes a sheath having a sheath body defining a lumen adapted to receive a dilator. The sheath body includes a proximal portion and a distal portion. A sheath hub is coupled to the proximal portion of the sheath. A dilator includes a dilator shaft defining a lumen adapted to receive and support a puncturing device. The dilator shaft includes a proximal portion for manipulation by a user and a tapered distal portion for placement in or near the heart. A dilator hub is coupled to the proximal portion of the dilator shaft. The dilator hub includes a rotational coupling structure for coupling to the sheath hub so as to inhibit relative rotation between the dilator and the sheath. An axial lock feature is configured to create a resistance to an axial disengagement force, such that the dilator hub is secured axially within the sheath hub.

Example 31 is the system of Example 30, wherein the sheath hub includes an opening having a tapered mating surface, and the dilator hub includes an angled disengagement surface adapted to contact the tapered mating surface.

Example 32 is the system of Example 30, wherein the rotational coupling structure includes a plurality of tapered surfaces.

Example 33 is the system of Example 30, wherein the axial lock feature includes an annular protrusion adapted to mate with a shoulder in the sheath hub.

Example 34 is a dilator hub for use with a dilator. The dilator hub includes a rotational coupling structure for coupling to a sheath hub so as to inhibit relative rotation between the dilator and a sheath. An axial lock feature is configured to create a resistance to an axial disengagement force, such that the dilator hub is secured axially within the sheath hub.

Example 35 is the dilator hub of Example 34, wherein the rotational coupling structure includes a plurality of tapered surfaces, and the axial lock feature includes an annular protrusion configured to mate with a shoulder in the sheath hub.

While multiple embodiments are disclosed, still other embodiments of the present invention will become apparent to those skilled in the art from the following detailed description, which shows and describes illustrative embodiments of the invention.

Accordingly, the drawings and detailed description are to be regarded as illustrative in nature and not restrictive.

### BRIEF DESCRIPTION OF THE DRAWINGS

FIGS. 1A-1C are schematic illustrations of a medical procedure within a patient's heart utilizing a transseptal access system according to embodiments of the invention.

FIGS. 2A-2B are perspective views of a dilator and sheath according to embodiments of the invention.

FIGS. 3A-3B are perspective views of a dilator inserted to a sheath (FIG. 3A) and a dilator hub engaging a sheath hub (FIG. 3B) according to embodiments of the invention.

FIG. 4A is a perspective view of a sheath hub and FIG. 4B is a plan view of a portion of a dilator hub mating with a sheath hub according to embodiments of the invention.

FIG. 5A is a sectional view of a dilator hub mating with a sheath hub and FIG. 5B is a perspective view of a dilator hub according to embodiments of the invention.

FIG. 6A is a sectional view of a dilator hub and FIG. 6B is a sectional view of a dilator hub mating with a sheath hub according to embodiments of the invention.

While the invention is amenable to various modifications and alternative forms, specific embodiments have been shown by way of example in the drawings and are described in detail below. The intention, however, is not to limit the invention to the embodiments described. On the contrary, the invention is intended to cover all modifications, equivalents, and alternatives falling within the scope of the invention as defined by the appended claims.

### DETAILED DESCRIPTION

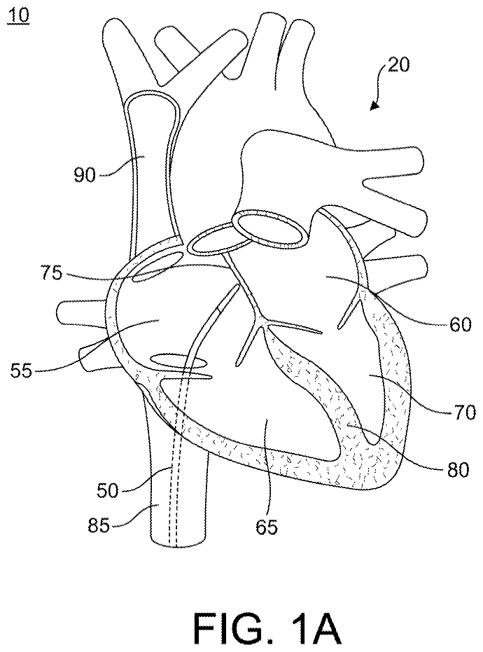

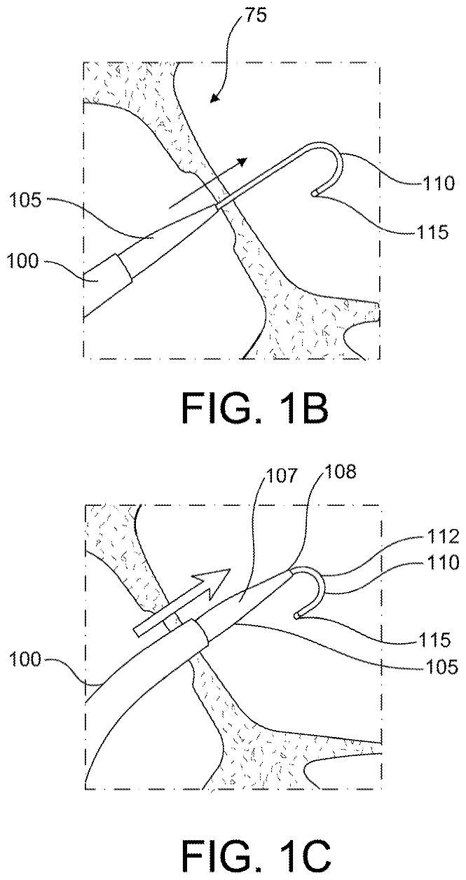

FIGS. 1A-1C are schematic illustrations of a medical procedure 10 within a patient's heart 20 utilizing a transseptal access system 50 according to embodiments of the disclosure. As is known, the human heart 20 has four chambers, a right atrium 55, a left atrium 60, a right ventricle 65 and a left ventricle 70. Separating the right atrium 55 and the left atrium 60 is an atrial septum 75 and separating the right ventricle 65 and the left ventricle 70 is a ventricular septum 80. As is further known, deoxygenated blood from the patient's body is returned to the right atrium 55 via an inferior vena cava (IVC) 85 or a superior vena cava (SVC) 90.

Various medical procedures have been developed for diagnosing or treating physiological ailments originating within the left atrium 60 and associated structures.

Exemplary such procedures include, without limitation, deployment of diagnostic or mapping catheters within the left atrium 60 for use in generating electroanatomical maps or diagnostic images thereof. Other exemplary procedures include endocardial catheter-based ablation (e.g., radiofrequency ablation, pulsed field ablation, cryoablation, laser ablation, high frequency ultrasound ablation, and the like) of target sites within the chamber or adjacent vessels (e.g., the pulmonary veins and their ostia) to terminate cardiac arrythmias such as atrial fibrillation and atrial flutter. Still other exemplary procedures may include deployment of left atrial appendage (LAA) closure devices. Of course, the foregoing examples of procedures within the left atrium 60 are merely illustrative and in no way limiting with respect to the present disclosure.

The medical procedure 10 illustrated in FIGS. 1A-1C is an exemplary embodiment for providing access to the left atrium 60 using the transseptal access system 50 for subsequent deployment of the aforementioned diagnostic and/or therapeutic devices within the left atrium 60. As shown in FIGS. 1A-1C, target tissue site can be defined by tissue on the atrial septum 75. In the illustrated embodiment, the target site is accessed via the IVC 85, for example through the femoral vein, according to conventional catheterization techniques. In other embodiments, access to the target site on the atrial septum 75 may be accomplished using a superior approach wherein the transseptal access system 50 is advanced into the right atrium 55 via the SVC 90.

In the illustrated embodiment, the transseptal access system 50 includes an introducer sheath 100, a dilator 105 having a dilator body 107 and a tapered distal tip portion 108, and a perforation device (e.g., a radiofrequency (RF) perforation device) 110 having distal end portion 112 terminating in a tip electrode 115. As shown, in the assembled use state illustrated in FIGS. 1A-1C, the RF perforation device 110 can be disposed within the dilator 105, which itself can be disposed within the sheath 100. In one embodiment in which the transseptal access system 50 is deployed into the right atrium 55 via the IVC 105, a user introduces a guidewire (not shown) into a femoral vein, typically the right femoral vein, and advances it towards the heart 20. The sheath 100 may then be introduced into the femoral vein over the guidewire, and advanced towards the heart 20. In one embodiment, the distal ends of the guidewire and sheath 100 are then positioned in the SVC 90. These steps may be performed with the aid of an imaging system, e.g., fluoroscopy or ultrasonic imaging. The dilator 105 may then be introduced into the sheath 100 and over the guidewire, and advanced through the sheath 100 into the SVC 90. Alternatively, the dilator 105 may be fully inserted into the sheath 100 prior to entering the body, and both may be advanced simultaneously towards the heart 20. When the guidewire, sheath 100, and dilator 105 have been positioned in the SVC 90, the guidewire is removed from the body, and the sheath 100 and the dilator 105 are retracted so that their distal ends are positioned in the right atrium 55. The RF perforation device 110 described can then be introduced into the dilator 105, and advanced toward the heart 20. In various embodiments, the guidewire and the RF perforation device are the same component, so an exchange is not necessary.

Subsequently, the user may position the distal end of the dilator 105 against the atrial septum 75, which can be done under imaging guidance. The RF perforation device 110 is then positioned such that electrode 115 is aligned with or protruding slightly from the distal end of the dilator 105. The dilator 105 and the RF perforation device 110 maybe dragged along the atrial septum 75 and positioned, for example against the fossa ovalis of the atrial septum 75 under imaging guidance. A variety of additional steps may be performed, such as measuring one or more properties of the target site, for example an electrogram or ECG (electrocardiogram) tracing and/or a pressure measurement, or delivering material to the target site, for example delivering a contrast agent. Such steps may facilitate the localization of the tip electrode 115 at the desired target site. In addition, tactile feedback provided by medical RF perforation device 110 is usable to facilitate positioning of the tip electrode 115 at the desired target site.

With the tip electrode 115 and dilator 105 positioned at the target site, energy is delivered from an energy source, e.g., an RF generator, through the RF perforation device 110 to the tip electrode 115 and the target site. In some embodiments, the energy is delivered at a power of at least about 5 W at a voltage of at least about 75 V (peak-to-peak), and functions to vaporize cells in the vicinity of the tip electrode 115, thereby creating a void or perforation through the tissue at the target site. The user then applies force to the RF perforation device 110 so as to advance the tip electrode 115 at least partially through the perforation. In these embodiments, when the tip electrode 115 has passed through the target tissue, that is, when it has reached the left atrium 60, energy delivery is stopped. In some embodiments, the step of delivering energy occurs over a period of between about 1 second and about 5 seconds.

With the tip electrode 115 of the RF perforation device 110 having crossed the atrial septum 75, the dilator 105 can be advanced forward, with the tapered distal tip portion 108 operating to gradually enlarge the perforation to permit advancement of the distal end of the sheath 100 into the left atrium 60.

In some embodiments, the distal end portion 112 of the RF perforation device 110 may be pre-formed to assume an atraumatic shape such as a J-shape (as shown in FIGS. 1B-1C), a pigtail shape or other shape selected to direct the tip electrode 115 away from the endocardial surfaces of the left atrium 60. Examples of such RF perforation devices can be found, for example, in U.S. patents application Ser. Nos. 16/445,790 and 16/346,404 assigned to Baylis Medical Company, Inc. The aforementioned pre-formed shapes can advantageously function to minimize the risk of unintended contact between the tip electrode 115 and tissue within the left atrium 60, and can also operate to anchor the distal end portion 112 within the left atrium 60 during subsequent procedural steps.

For example, in embodiments, the RF perforation device 110 can be structurally configured to function as a delivery rail for deployment of a relatively larger bore therapy delivery sheath and associated dilator(s). In such embodiments, the dilator 105 and the sheath 100 are withdrawn following deployment of the distal end portion 112 of the RF perforation device 110 into the left atrium 60. The anchoring function of the pre-formed distal end portion 112 inhibits unintended retraction of the distal end portion 112, and corresponding loss of access to the perforated site on the atrial septum 75, during such withdrawal.

Various medical procedures have been developed for diagnosing or treating physiological ailments originating within the left atrium 60 and associated structures. Exemplary such procedures include, without limitation, deployment of diagnostic or mapping catheters within the left atrium 60 for use in generating electroanatomical maps or diagnostic images thereof. Other exemplary procedures include endocardial catheter-based ablation (e.g., radiofrequency ablation, pulsed field ablation, cryoablation, laser ablation, high frequency ultrasound ablation, and the like) of target sites within the chamber or adjacent vessels (e.g., the pulmonary veins and their ostia) to terminate cardiac arrythmias such as atrial fibrillation and atrial flutter. Still other exemplary procedures may include deployment of left atrial appendage (LAA) closure devices. Of course, the foregoing examples of procedures within the left atrium 60 are merely illustrative and in no way limiting with respect to the present disclosure.

In certain embodiments, catheters, therapy devices and sheaths can be deployed through the sheath 100, after it is successfully deployed into the desired heart chamber (e.g., the left atrium). In other embodiments, the therapy device (e.g., mapping catheter, therapy sheath, medical device, etc.) is part of the sheath 100, creating a therapy sheath.

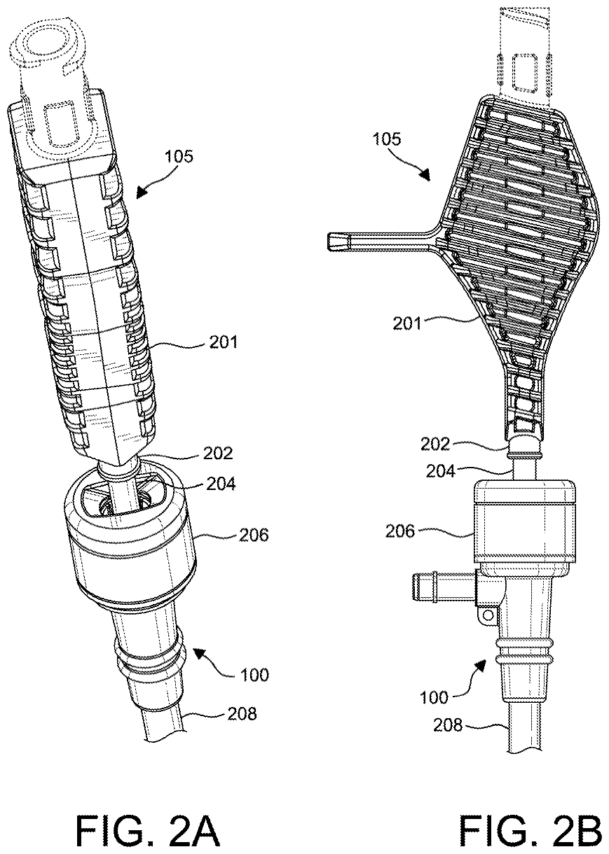

FIGS. 2A-2B are perspective views of a dilator and sheath according to embodiments of the invention. As shown, the dilator 105 is partially inserted into a lumen of the sheath 100. The dilator 105 includes a handle 201, a dilator hub 202 and a dilator shaft 204. The sheath 100 includes a sheath hub 206 and a sheath body 208.

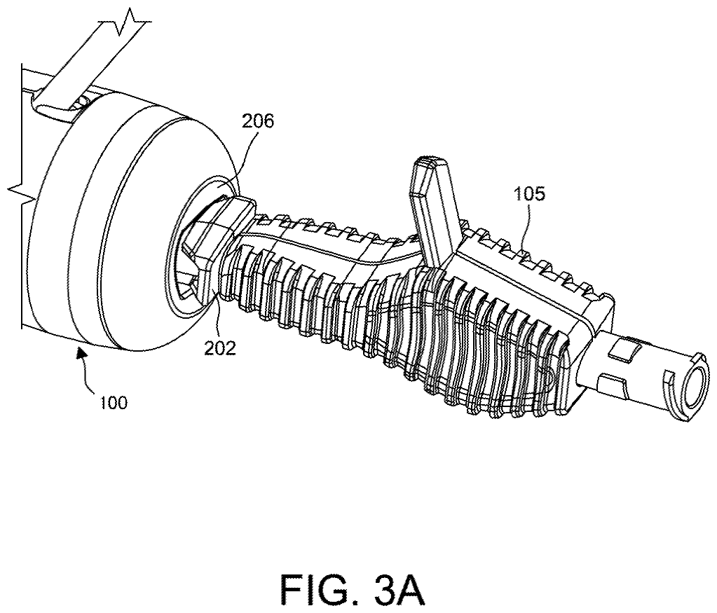

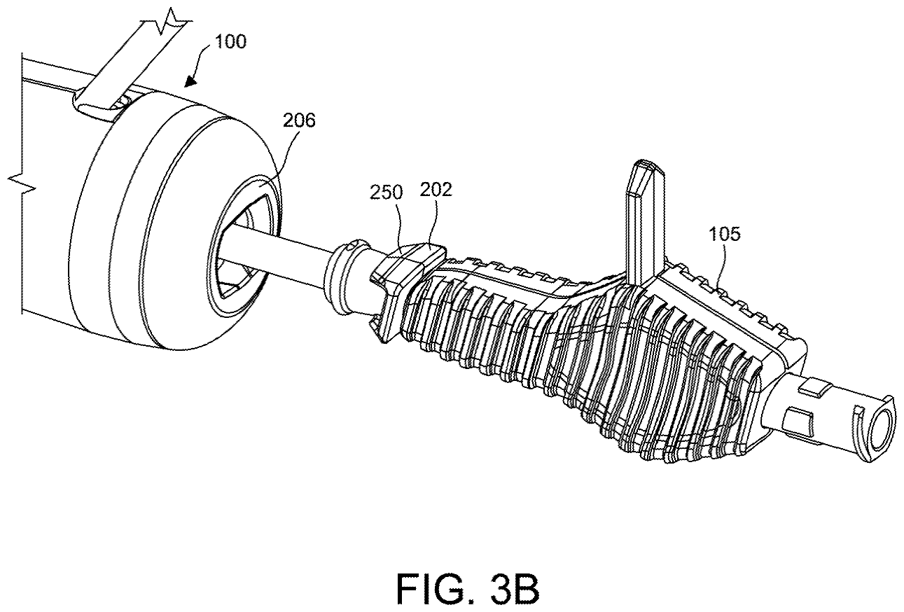

FIGS. 3 are perspective views of a dilator inserted into a sheath such that the dilator hub 202 begins to interact with the sheath hub 206 (FIG. 3A) and a dilator partially inserted into a sheath (FIG. 3B) according to embodiments of the invention. As shown, the dilator hub 202 is adapted to mate with the sheath hub 206. The hub 202 and 206 are shaped so as to inhibit or prevent relative rotation of the dilator 105 with respect to the sheath 100 when the dilator hub 202 is fully inserted into the sheath hub 206. As shown in FIG. 3A, the tapered surfaces on the hubs 202, 206 are sized and shaped to allow the user to begin inserting the dilator 105 at a certain offset angle from the longitudinal. Likewise, the user can insert the dilator at a certain rotational offset angle from the final engagement angle. These features facilitate the user in inserting and engaging the mating hubs, as the insertion angle does not need to be perfectly aligned. In these embodiments, the tapered surfaces will guide the dilator hub 202 into the proper engagement orientation. As shown, the surfaces will self-align from an offset angle as great as 20 degrees. Once the dilator hub 202 is positioned within the sheath hub 206, relative rotation of the dilator 105 with respect to the sheath 100 is prevented and a rotational lock between the two components is created. This allows a user to simultaneously rotate the dilator 105 and sheath 100 together.

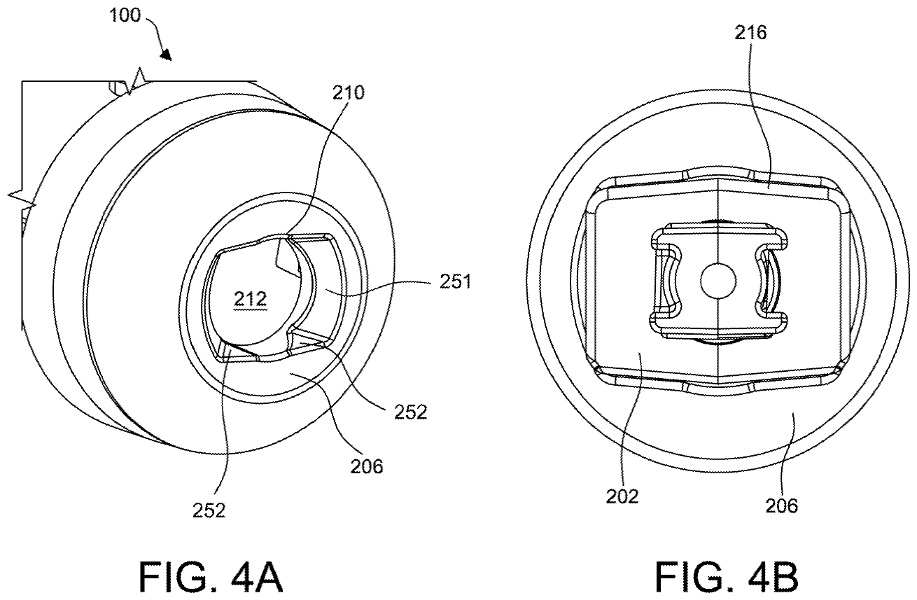

FIG. 4A is a perspective view of a sheath hub and FIG. 4B is a plan view of a portion of a dilator hub mating with a sheath hub according to embodiments of the invention. As shown in FIG. 4A, the sheath hub 206 has an opening 210 surrounding a lumen 212 adapted to accept the dilator shaft. In various embodiments the opening 210 has a tapered or funnel shape in the axial direction. The opening 210 includes arcuate angled or tapered surfaces 251 on opposite sides of the lumen 212. The opening 210 also includes tapered surfaces 252 located adjacent the arcuate tapered surfaces 251 on the bottom and top of the opening 210. In one aspect there are four tapered surfaces 252. As discussed further below, the tapered surfaces 252 are configured to interact with angled disengagement surfaces 250 of the dilator hub 202. In certain embodiments, the arcuate tapered surfaces 251 are configured to interact with angled disengagement surfaces 250 to As shown in FIG. 4B, upon longitudinal insertion of the dilator 105 into the sheath, the dilator hub 202 has an outer surface 216 that remains outside of the opening 210 in the sheath hub 206, while the tapered surfaces 252 and the angled disengagement surfaces 250 are in contact with one another. In this mated configuration, relative rotation of the sheath hub 206 and the dilator hub 202 is resisted. The outer surface 216 includes opposing longitudinal surfaces 230 and 232, which extend along the longitudinal axis of the hub, and opposing lateral surfaces 240 and 242, which extend radially outward and generally perpendicular to the longitudinal axis.

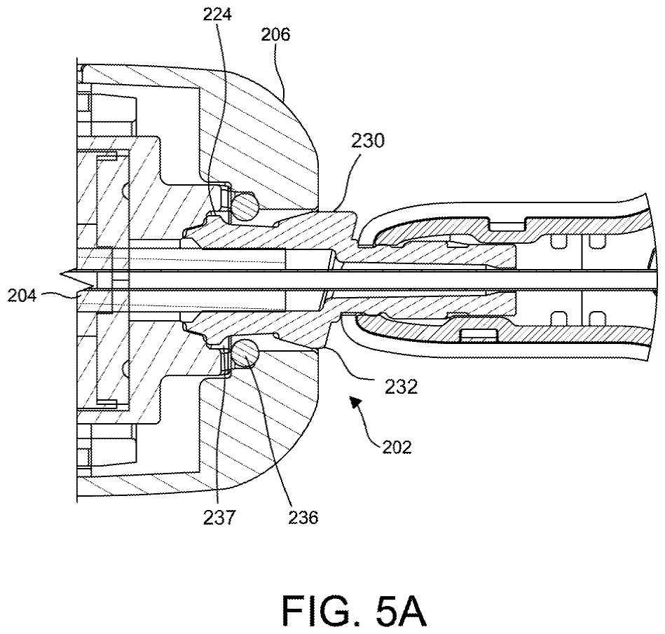

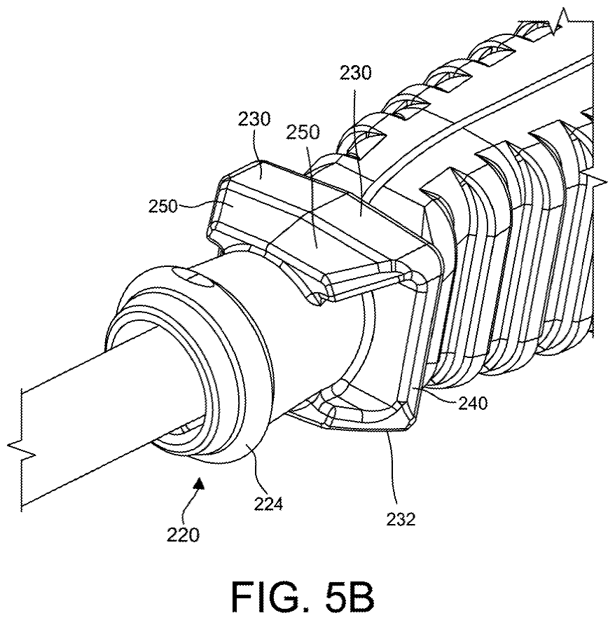

FIG. 5A is a sectional view of a dilator hub 202 mating with a sheath hub 206 and FIG. 5B is a perspective view of a dilator hub 202 according to embodiments of the invention. As shown in FIG. 5A, the dilator is fully inserted into the sheath, such that the dilator hub 202 is fully engaged with the sheath hub 206. As shown, the distal end of the dilator hub 202 includes an axial lock feature 220, which includes a protrusion 224. In some aspects, the protrusion 224 is an annular protrusion located on an outer circumference of the dilator hub 202. As shown in FIG. 5A, these protrusions 224 are adapted to engage with corresponding shoulders 236 on the sheath hub 206. The interaction between the protrusions 224 and the shoulders 236 creates a resistance to an axial disengagement force, such that the dilator hub 202 is secured axially within the sheath hub 206. In one aspect, the shoulders 236 can take the form of an elastic and deformable o-ring. Upon insertion of the dilator hub 202 into the sheath hub 206, the protrusions 224 deform the o-ring and enter into a locking chamber 237. The protrusions 224 remain within the locking chamber 237, unable to move proximally past the shoulders 236, in order to axial lock the dilator hub 202 to the sheath hub 206. In one aspect, the locking chamber 237 is configured to allow slight axial movement of the protrusions 224 by providing space between the shoulders 236 and the protrusions 224. In another aspect, the locking chamber 237 is configured to securely hold protrusions 224, wherein the protrusions are in contract with the shoulders 236. The dilator hub 202 and the sheath hub 206 are configured such that when the protrusions are positioned within the locking chamber 237, the tapered surfaces 252 and the angled disengagement surfaces 250 are in close proximity to or in contact with one another, so as to resist or prevent relative rotation as discussed above.

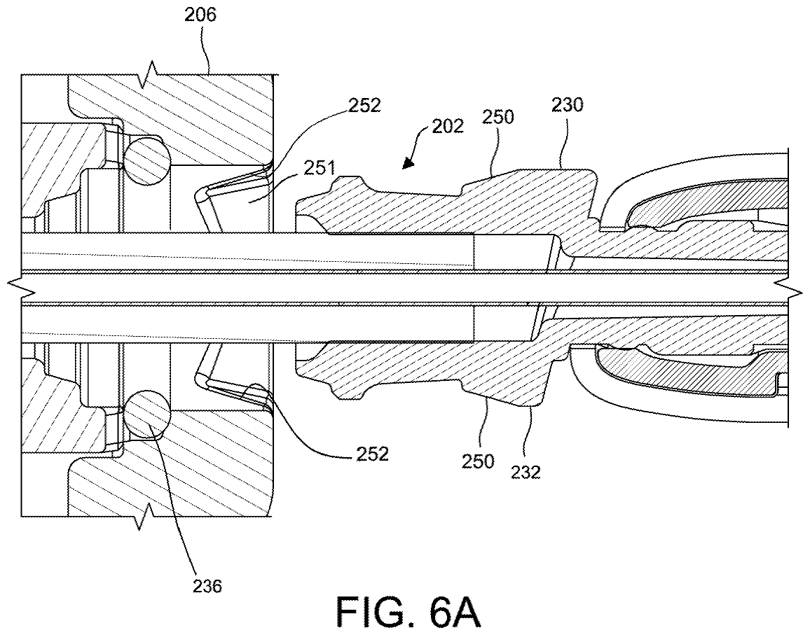

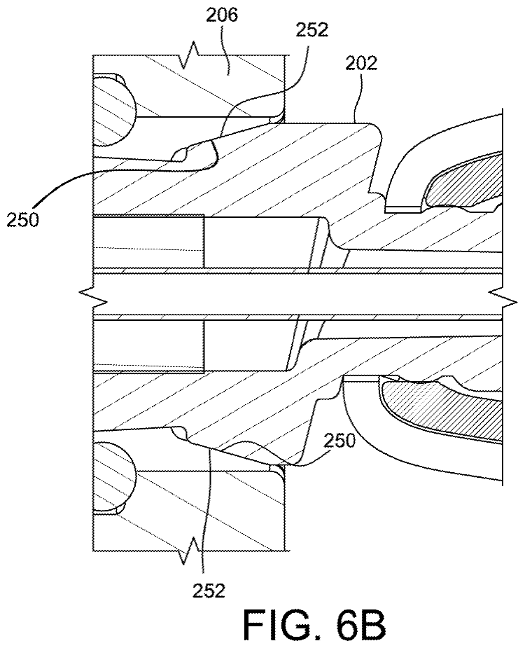

FIG. 6A is a sectional view of a dilator hub and FIG. 6B is a sectional view of a dilator hub mating with a sheath hub according to embodiments of the invention. As shown, the dilator hub 202 includes an angled disengagement surface 250. This surface 250 mates with a corresponding tapered surface 252 on the sheath hub 206. The two surfaces may have slightly different angles or the same angle (but not oriented horizontally), such that upon application of a torque to the dilator hub 202, the surface 252 generates an axial disengagement force upon the disengagement surface 250. In one aspect, the angled disengagement surface 250 and the tapered surface 252 may have an angle with respect to the longitudinal axis in the range of 5 degrees to 25 degrees. In one aspect, the angle may be approximately 10 degrees. The disengagement force (having an axial force component) causes an axial motion of the dilator hub 202, which disengages the dilator hub 202 from the sheath hub 206. When the disengagement force becomes sufficient to overcome an axial lock force formed by an interaction with the annular protrusions 224 and the shoulders 236, relative motion in both axial and rotational directions occurs. By changing the angles of these surfaces, the amount of torque and the degree of rotation required for disengagement may be adjusted.

In certain embodiments, the surface 250 mates with a corresponding arcuate tapered surface 251 on the sheath hub 206. The two surfaces may have slightly different angles or the same angle (but not oriented horizontally), such that upon application of a torque to the dilator hub 202, the surface 251 generates an axial disengagement force upon the disengagement surface 250. In one aspect, the angled disengagement surface 250 and the surface 251 may have an angle with respect to the longitudinal axis in the range of 5 degrees to 25 degrees. In one aspect, the angle may be approximately 10 degrees. The disengagement force causes an axial motion of the dilator hub 202, which disengages the dilator hub 202 from the sheath hub 206. When the disengagement force becomes high enough to overcome an axial lock formed by an interaction with the annular protrusions 224 and the shoulders 236, relative motion in both axial and rotational directions occurs. By changing the angles of these surfaces, the amount of torque and the degree of rotation required for disengagement may be adjusted. In some embodiments, both surfaces 251 and 252 are configured to interact with surface 250 to generate the disengagement force.

Various modifications and additions can be made to the exemplary embodiments discussed without departing from the scope of the present invention. For example, while the embodiments described above refer to particular features, the scope of this invention also includes embodiments having different combinations of features and embodiments that do not include all of the described features. Accordingly, the scope of the present invention is intended to embrace all such alternatives, modifications, and variations as fall within the scope of the claims, together with all equivalents thereof.

I claim:

1. A dilator for facilitating access to a patient's heart and for coupling with a sheath including a sheath hub, the dilator comprising:
a dilator shaft defining a lumen adapted to receive and support a puncturing device, the dilator shaft includes a proximal portion for manipulation by a user and a tapered distal portion for placement in or near the heart; and
a dilator hub coupled to the proximal portion of the dilator shaft, the dilator hub including a rotational coupling structure for coupling to the sheath hub so as to inhibit relative rotation between the dilator and the sheath;
wherein the dilator hub is configured to self-align into proper engagement with the sheath hub.

2. The dilator of claim 1, wherein the dilator hub is directly coupled to the dilator shaft.

3. The dilator of claim 1, wherein the sheath is a therapy sheath and the puncturing device is an RF puncturing device.

4. The dilator of claim 1, further comprising an axial lock feature configured to create a resistance to an axial disengagement force, such that the dilator hub is secured axially within the sheath hub.

5. The dilator of claim 1, wherein the axial lock feature includes a protrusion adapted to mate with a shoulder.

6. The dilator of claim 5, wherein the protrusion is annular.

7. The dilator of claim 1, wherein the dilator hub includes an angled disengagement surface adapted to contact a mating surface on the sheath hub.

8. The dilator of claim 7, wherein upon rotation of the dilator hub, the mating surface generates an axial disengagement force upon the disengagement surface.

9. The dilator of claim 8, wherein when the disengagement force becomes high enough to overcome an axial lock, relative motion in both axial and rotational directions occurs.

10. The dilator of claim 7, wherein the angled disengagement surface and the mating surface have a different angle.

11. The dilator of claim 7, wherein the angled disengagement and the mating surface have a same angle.

12. The dilator of claim 1, wherein the rotational coupling structure includes a plurality of tapered surfaces.

13. The dilator of claim 12, wherein the sheath hub includes surfaces corresponding to the plurality of tapered surfaces.

14. The dilator of claim 1, wherein the sheath hub has a tapered or funnel shape opening.

15. A system for facilitating access to a patient's heart, the system comprising:
a sheath having a sheath body defining a lumen adapted to receive a dilator, the sheath body including a proximal portion and a distal portion;
a sheath hub coupled to the proximal portion of the sheath;
a dilator having a dilator shaft defining a lumen adapted to receive and support a puncturing device, the dilator shaft including a proximal portion for manipulation by a user and a tapered distal portion for placement in or near the heart;
a dilator hub coupled to the proximal portion of the dilator shaft, the dilator hub comprising:
a rotational coupling structure for coupling to the sheath hub so as to inhibit relative rotation between the dilator and the sheath; and
an axial lock feature configured to create a resistance to an axial disengagement force, such that the dilator hub is secured axially within the sheath hub.

16. The system of claim 15, wherein the sheath hub includes an opening having a tapered mating surface, and the dilator hub includes an angled disengagement surface adapted to contact the tapered mating surface.

17. The system of claim 15, wherein the rotational coupling structure includes a plurality of tapered surfaces.

18. The system of claim 15, wherein the axial lock feature includes an annular protrusion adapted to mate with a shoulder in the sheath hub.

19. A dilator hub for use with a dilator, the dilator hub comprising:
a rotational coupling structure for coupling to a sheath hub so as to inhibit relative rotation between the dilator and a sheath; and
an axial lock feature configured to create a resistance to an axial disengagement force, such that the dilator hub is secured axially within the sheath hub.

20. The dilator hub of claim 19, wherein the rotational coupling structure includes a plurality of tapered surfaces, and the axial lock feature includes an annular protrusion configured to mate with a shoulder in the sheath hub.
